(19) United States
(12) Patent Application Publication
(10) Pub. No.: US 2025/0235322 A1
(43) Pub. Date: Jul. 24, 2025

(54) METATARSOPHALANGEAL ARTHROPLASTY PROSTHESIS SYSTEM AND METHOD

(71) Applicant: BON SECOURS MERCY HEALTH, INC., Cincinnati, OH (US)

(72) Inventors: Nick Nadaud, Perrysburg, OH (US); David Michael Uhlenhake, Toledo, OH (US)

(21) Appl. No.: 18/852,658

(22) PCT Filed: Apr. 3, 2023
(86) PCT No.: PCT/US2023/065272

§ 371 Date: Sep. 30, 2024

Related U.S. Application Data
(60) Provisional application No. 63/326,379, filed on Apr. 1, 2022.

(51) Int. Cl. A61F 2/42; A61B 17/72; A61F 2/30

(52) U.S. Cl. CPC A61F 2/4225; A61B 17/7233; A61B 17/7291; A61F 2002/30112; A61F 2002/4233; A61F 2310/00023

(57) ABSTRACT

A metatarsophalangeal arthroplasty prothesis system can include a metatarsal post screw including a cross hole formed therethrough. The system can include a metatarsal cross screw configured to be disposed in the cross hole of the metatarsal post screw. The system can include a metatarsal guide configured to be removably disposed in the metatarsal post screw and configured to guide the metatarsal cross screw into the cross hole. The system can include a metatarsal cap configured to be removably disposed in the metatarsal post screw. The system can include a toe post screw including a cross hole formed therethrough. The system can include a toe cross screw configured to be disposed in the cross hole. The system can include a toe guide and a toe cap configured to be removably disposed in the toe post screw.

### CROSS-REFERENCE TO RELATED APPLICATIONS

This application claims the benefit of U.S. Provisional Application No. 63/326,379, filed on Apr. 1, 2022. The entire disclosure of the above application is incorporated herein by reference.

### FIELD

The present technology includes processes and articles of manufacture that relate to metatarsophalangeal arthroplasty joint replacement, including metatarsophalangeal arthroplasty joint replacement implants.

### INTRODUCTION

This section provides background information related to the present disclosure which is not necessarily prior art.

Damage to the first metatarsal-phalangeal (MTP) joint, where the base of the big toe meets the foot may cause pain and stiffness in the joint, and with time, it may become increasingly harder to bend the toe. Some symptoms of a damaged MTP joint may include pain and stiffness in the big toe during use (e.g., walking, standing, bending, etc.), pain and stiffness that is aggravated by cold, damp weather, difficulty with certain activities (e.g., running, squatting), and swelling and inflammation around the joint. Common causes of damage to the MTP joint may include faulty function (biomechanics) and structural abnormalities of the foot, genetics, overuse, and injury, such as stubbing the big toe. Damage to the MTP joint may also be caused by inflammatory diseases, such as rheumatoid arthritis or gout.

In many cases, early treatment may prevent or postpone the need for surgery. Such conservative treatment for mild or moderate damage to the MTP joint may include shoe modification, orthotic devices, medication, injection therapy, and physical therapy. However, in some cases, surgery may be the only way to eliminate or reduce pain. Surgical options may include a traditional approach where two ends of the joint are screwed together, replacement of the cartilage of the joint, a flexible hinge implant or replacement, and a fusion of the MTP joint.

In certain cases, an implant may be used to cover damaged or missing articular cartilage in the MTP joint, where the base of the big toe meets the foot. The implant may restore mobility to the bones of this joint, allowing them to glide smoothly against each other. Although, implants have improved, they are often not the first option for many podiatrists and surgeons. For an implant to be successful it is desirable that the implant be robust and durable, easy to replace as the components wear, minimize the amount of bone removal, and be removable if a fusion of the joint is necessary. Current implants, however, may fracture unexpectedly, may cause joint stiffness, may suffer from misalignment and/or poor positioning, may cause damage to the bone, and may cause a material reaction with the body.

Accordingly, there is a need for an implant that minimizes bone loss, prevents regression, is removable and/or replaceable, and is designed so that fusion of the joint may still occur if the implant is removed.

### SUMMARY

In concordance with the instant disclosure, an implant that minimizes bone loss, prevents regression, is removable and/or replaceable, and is designed in a way that a fusion of the joint may still occur if the implant is removed, is surprisingly discovered.

In one embodiment, a metatarsophalangeal arthroplasty prothesis system can include a metatarsal post screw including a cross hole formed therethrough. The system can include a metatarsal cross screw configured to be disposed in the cross hole of the metatarsal post screw. The system can include a metatarsal guide configured to be removably disposed in the metatarsal post screw and configured to guide the metatarsal cross screw into the cross hole. The system can include a metatarsal cap configured to be removably disposed in the metatarsal post screw. The system can include a toe post screw including a cross hole formed therethrough. The system can include a toe cross screw configured to be disposed in the cross hole. The system can include a toe guide and a toe cap configured to be removably disposed in the toe post screw.

In another embodiment, a method of installing a metatarsophalangeal arthroplasty prothesis system, includes: providing the metatarsophalangeal arthroplasty prothesis system including: a metatarsal post screw including a cross hole formed therethrough; a metatarsal cross screw configured to be disposed in the cross hole of the metatarsal post screw; a metatarsal guide configured to be removably disposed in the metatarsal post screw and configured to guide the metatarsal cross screw into the cross hole of the metatarsal cross screw; a metatarsal cap configured to be removably disposed in the metatarsal post screw; a toe post screw including a cross hole formed therethrough; a toe cross screw configured to be disposed in the cross hole of the toe post screw; a toe guide configured to be removably disposed in the toe post screw and configured to guide the toe cross screw into the cross hole of the toe post screw; and a toe cap configured to be removably disposed in the toe post screw; accessing and disarticulating the metatarsophalangeal joint inserting a guide wire into the center and along a length of one of the metatarsal bone or the corresponding proximal phalanx; drilling a bore for one of the metatarsal post screw and the toe post screw; inserting the metatarsal post screw and the toe post screw into the prepared bore; inserting one of the metatarsal guide and the toe guide; installing the cross screw placing one of the metatarsal cap and the toe cap.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a top perspective view of a metatarsophalangeal arthroplasty prothesis system, according to one embodiment of the present disclosure;

FIG. 2 is a cross-sectional, side elevational view of the metatarsophalangeal arthroplasty prothesis system, taken at line 2-2 in FIG. 1;

FIG. 3 is a top perspective view of the metatarsophalangeal arthroplasty prothesis system of FIG. 1, further depicting guides for placing the system, according to some embodiments of the present disclosure;

FIG. 4 is a top perspective view of a metatarsal cap of the metatarsophalangeal arthroplasty prothesis system of FIG. 1;

FIG. 5 is a front perspective view of the metatarsophalangeal arthroplasty prothesis system of FIG. 1, further depicting the system without a cap or a guide;

FIG. 6 is a top perspective view of a metatarsal guide of the metatarsophalangeal arthroplasty prothesis system of FIG. 3;

FIG. 7 is a top plan view of the metatarsophalangeal arthroplasty prothesis system installed within the metatarsal bone and the corresponding proximal phalanx of a foot of a patient;

FIG. 8 is a side elevational view of the metatarsophalangeal arthroplasty prothesis system during installation; and

FIG. 9 is a flow chart depicting a method of installing the metatarsophalangeal arthroplasty prothesis system, according to one embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps may be different in various embodiments, including where certain steps may be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping, or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology relates to metatarsophalangeal arthroplasty joint replacement implants. The present technology includes an implant for the metatarsalphalangeal (MTP) joint that minimizes bone loss, prevents regression, is removable and/or replaceable, and is designed to be removed for a fusion of the joint. As described herein, the implant can be used in any metatarsalphalangeal joint, but is specifically shown in the first metatarsalphalangeal joint. The relative sizes of the implant can vary based both on the size of the joint to be treated as well as the individual into which the implant is being placed.

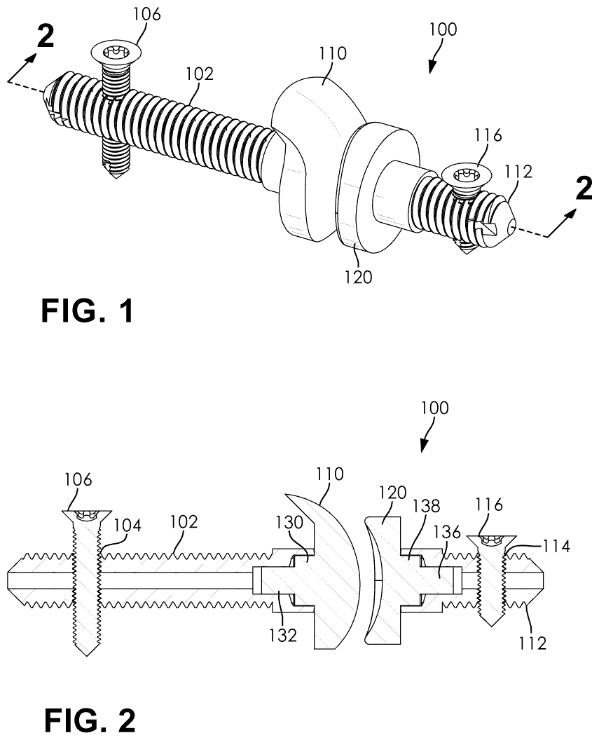

As shown in FIGS. 1-8, a metatarsophalangeal arthroplasty prothesis system 100 can include a metatarsal post screw 102, which can include a cross hole 104 formed therethrough. A metatarsal cross screw 106 can be configured to be disposed in the cross hole 104 of the metatarsal post screw 102. A metatarsal guide 108 can be configured to be removably disposed in the metatarsal post screw 102 and be configured to guide the metatarsal cross screw 106 into the cross hole 104 of the metatarsal post screw 102. A metatarsal cap 110 can be configured to be removably disposed in the metatarsal post screw 102. A toe post screw 112 can include a cross hole 114 formed therethrough. A toe cross screw 116 can be configured to be disposed in the cross hole 114 of the toe post screw 112. A toe guide 118 can be configured to be removably disposed in the toe post screw 112 and can be configured to guide the toe cross screw 116 into the cross hole 114 of the toe post screw 112. A toe cap 120 can configured to be removably disposed in the toe post screw 112.

Each of the metatarsal post screw 102 and the toe post screw 112 can be self-tapping screws. Each of the metatarsal post screw 102 and the toe post screw 112 can be cannulated to receive a guide wire during installation, as described in greater detail herein. The metatarsal post screw 102 can be configured to be inserted into a metatarsal bone 101 of a foot of a patient. The toe post screw 112 can be configured to be inserted into a corresponding proximal phalanx 103 of the foot of the patient. Specifically, the metatarsal bone 101 of the phalanx 103 of the foot of the patient can be from a big toe of the patient. However, the metatarsal post screw 102 and the toe post screw 112 can be inserted in any metatarsal bone 101 and proximal phalanx 103 as deemed medically necessary by a skilled artisan.

Each of the metatarsal post screw 102 and the toe post screw 112 can be manufactured from titanium or other biocompatible materials having similar tensile strength, as non-limiting examples. Each of the metatarsal post screw 102 and the toe post screw 112 can be a M6 bolt, as a non-limiting example. Each of the metatarsal post screw 102 and the toe post screw 112 can have a length based on the relative size of the metatarsal bone 101 and the corresponding proximal phalanx of the patient. For illustration purposes, when being implanted into the first metatarsalphalangeal joint, metatarsal post screw 102 can have a length of 30 mm, and the toe post screw 112 can have a length of 12 mm, as non-limiting examples. A skilled artisan can select other suitable bolt sizes and lengths for the post screws 102, 112, as desired. The size of the implant 100, and the relates metatarsal post screw 102 and toe post screw can vary based on the metatarsalphalangeal joint into which the implant is being placed, as well as the size of the existing joint. This enables the implant to be universally utilized regardless of patient size and/or joint damage.

Each of the metatarsal post screw 102 and the toe post screw 112 can include a star head formed therein. Accordingly, the metatarsal post screw 102 and the toe post screw 112 can be installed with a star driver. In one particular non-limiting embodiment, the metatarsal post screw 102 and the toe post screw 112 can have a star head formed therein that is configured to be compatible with a T-30 star driver, such that a star driver can be inserted into the star head for placement of the metatarsal post screw 102 and toe post screw 112. A skilled artisan can select other suitable shapes and sizes for a head within each of metatarsal post screw 102 and the toe post screw 112, as desired, thus enabling placement of the metatarsal post screw 102 and toe post screw 112 with alternative drivers.

It should be appreciated each that the use of M6 bolts for each of the metatarsal post screw 102 and the toe post screw 112 can militate against bone loss of the patient after installation into the foot of the patient compared to other known prothesis systems.

As described herein, the metatarsal post screw 102 can include a cross hole 104 configured to receive the metatarsal post screw 102 and the toe post screw 112 can include a cross hole 114 configured to receive the toe cross screw 116. Accordingly, each of the cross holes 104, 114 can be threaded. Advantageously, the threads of the cross holes 104, 114 can allow for the cross screws 104, 114 to be aligned during installation. Further, the cross hole 104, 114 and cross screws 104, 114 prevent the post screws 102, 112 from backing out.

Each of the metatarsal cross screw 106 and the toe cross screw 116 can be configured to secure the post screws 102, 112 in place in the foot of the patient. Each of the metatarsal cross screw 106 and the toe cross screw 116 can be manufactured from titanium, as a non-limiting example. Each of the metatarsal cross screw 106 and the toe cross screw 116 can be countersunk, self-tapping bolts. Each of the metatarsal cross screw 106 and the toe cross screw 116 can be M3 bolts, as non-limiting examples. Each of the metatarsal cross screw 106 and the toe cross screw 116 can have a length based on the relative size of the metatarsal bone 101 and the corresponding proximal phalanx 103 of the patient. The metatarsal cross screw 106 can have a length of 16 mm, and the toe cross screw 116 can have a length of 10 mm, as non-limiting examples. A skilled artisan can select other suitable bolt sizes and lengths for the cross screws 106, 116, as desired based on the size of the joint to be treated.

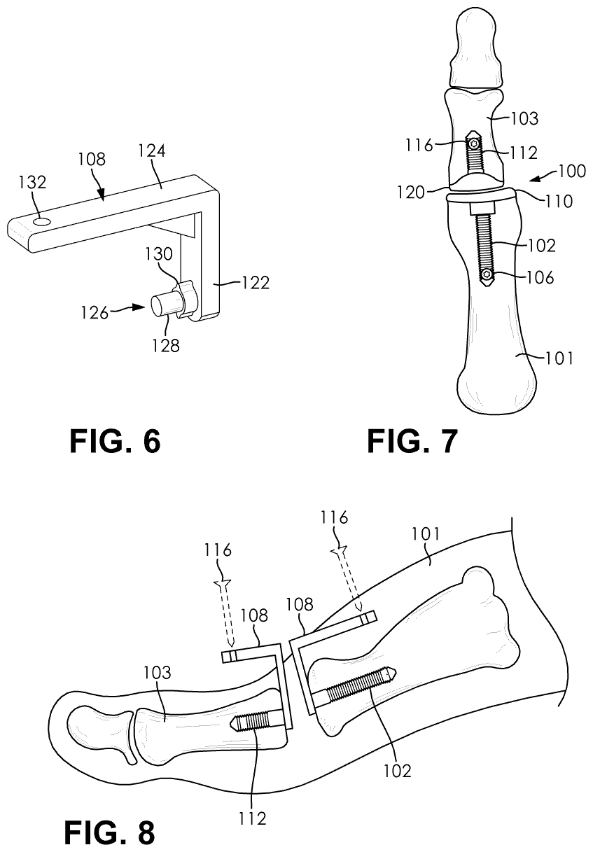

As shown in FIGS. 3 and 6, the metatarsal guide 108 can be configured to be selectively, removably press fit into the star head of the metatarsal post screw 102 and the toe guide 118 can be configured to be selectively, removably press fit into the star head of the toe post screw 112. Each of the metatarsal guide 108 and the toe guide 118 can be manufactured from stainless steel, as a non-limiting example. Each of the metatarsal guide 108 and the toe guide 118 can be substantially L-shaped including a first arm 122 and a second arm 124. The first arm 122 can be disposed at a 90-degree angle relative to the second arm 124.

During installation, and as shown in FIGS. 3 and 8, the first arm 122 of the metatarsal guide 108 is substantially perpendicular to the metatarsal post screw 102 and the second arm 124 of the metatarsal guide 108 is substantially parallel to the metatarsal post screw 102 when the metatarsal guide 108 is disposed on the star head of the metatarsal post screw 102. Similarly, the first arm 122 of the toe guide 118 is substantially perpendicular to the toe post screw 112 and the second arm 124 of the toe guide 118 is substantially parallel to the toe post screw 112 when the toe guide 118 is disposed on the star head of the toe post screw 112.

The first arm 122 of each of the metatarsal guide 108 and the toe guide 118 can include an attachment assembly 126, for example, as shown in FIG. 6. The attachment assembly 126 can include a post 128 and notch 130 that are configured to mate with and to provide an interference fit with the star head of each of the metatarsal post screw 102 and the toe post screw 112. In particular, the posts 128 can mate with the cannula of the post screws 102, 112 to align the guides 108, 118 within the post screws 102, 112. The notch 130 can be configured to mate within the star head of the post screws 102, 112. Advantageously, the notch can militate against rotational movement of the guides 108, 118, in operation.

The second arm 124 of each of the metatarsal guide 108 and the toe guide 118 can include a guide hole 132, for example, as shown in FIG. 6. The guide hole 132 of the metatarsal guide 108 can be configured to be aligned the cross hole 104 of the metatarsal post screw 102 when the metatarsal guide 108 is disposed on the metatarsal post screw 102. Similarly, the guide hole 132 of the toe guide 118 can be configured to be aligned the cross hole 114 if the toe post screw 112 when the toe guide 118 is disposed on the toe post screw 112. In certain embodiments, the guide hole 132 of the metatarsal guide and the guide hole of the toe guide are threaded. Advantageously, the threads of the guide holes 132 can allow for the cross screws 104, 114 to be aligned during installation.

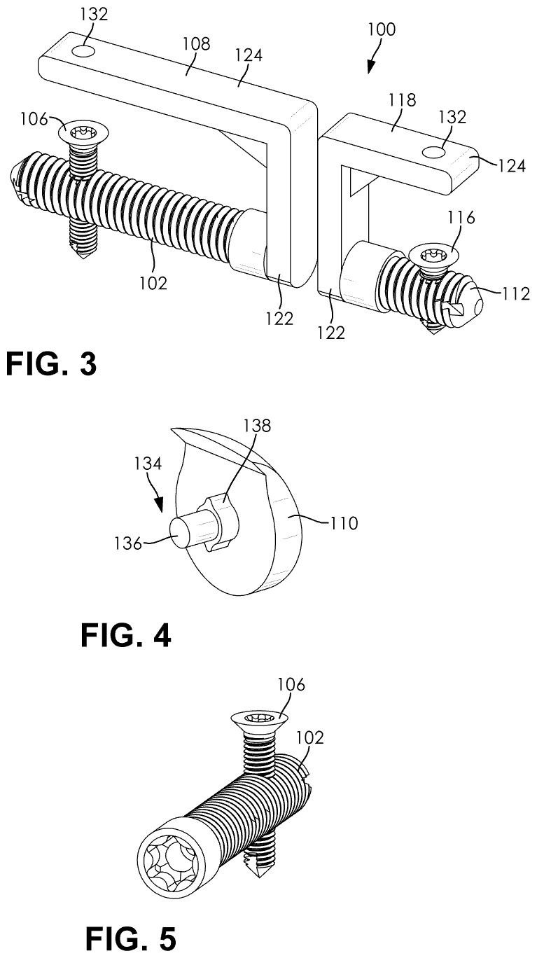

As shown in FIGS. 1-2 and 4, the metatarsal cap 110 can be configured to be selectively, removably press fit into the star head of the metatarsal post screw 102 and the toe cap 120 can be configured to be selectively, removably press fit into the star head of the toe post screw 112. The caps 110, 120 can form a seal with the corresponding post screw 102, 112, which can advantageously militate against undesirable bone growth around the metatarsophalangeal arthroplasty prothesis system 100. Each of the metatarsal cap 110 and toe cap 120 can include an attachment assembly 134, for example, as shown in FIG. 4. The attachment assembly 134 can include a post 136 and notch 138 that are configured to mate with and to provide an interference fit with the star head of each of the metatarsal post screw 102 and the toe post screw 112. In particular, the posts 136 can mate with the cannula of the post screws 102, 112 to align the caps 110, 120 within the post screws 102, 112. The notch 138 can be configured to mate within the star head of the post screws 102, 112. Advantageously, the notch 138 can militate against rotational movement of the caps 110, 120, in operation.

The metatarsal cap 110 can be manufactured from cobalt chrome, as a non-limiting example. The metatarsal cap 110 can have a smooth convex face. The toe cap 120 can be manufactured from titanium, as a non-limiting example. The toe cap 120 can have a concave face configured to receive the convex face of the metatarsal cap 110. The face of the toe cap 120 can be covered with nylon, as a non-limiting example. Advantageously, the nylon can militate against wear to the caps 110, 120 over time as the patient utilizes the first metatarsophalangeal arthroplasty prothesis system 100.

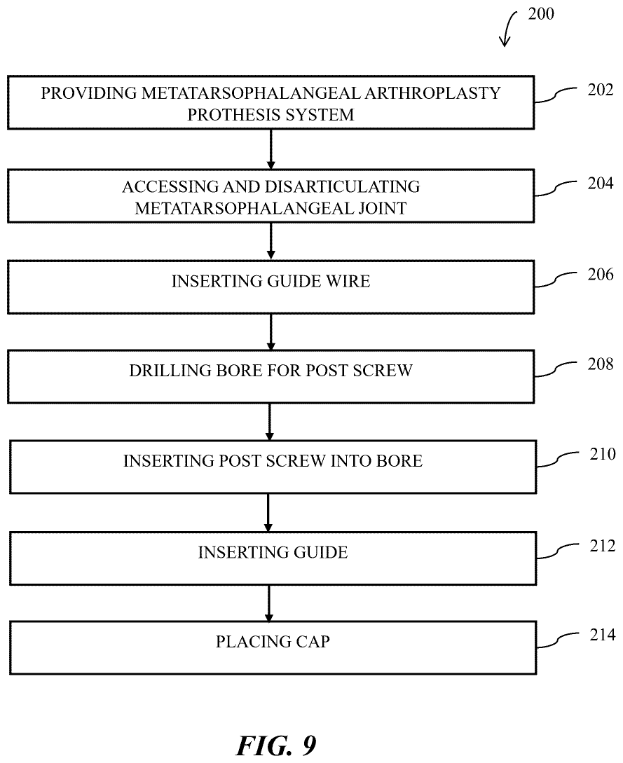

The present disclosure further contemplates a method 200 of installing the metatarsophalangeal arthroplasty prothesis system 100, for example as shown in FIG. 9. For exemplar purposes, the method is described with regard to a first metatarsophalangeal arthroplasty. The method 200 can include a step 202 providing the first metatarsophalangeal arthroplasty prothesis system. A step 204 can include accessing the MTP joint. The step 204 can include making a dorsal incision along the top of the foot of the patient and disarticulating the joint. The method 200 can include a step 206 of inserting a guide wire into the center and along a length of one of the metatarsal bone 101 or the corresponding proximal phalanx 103 of the foot of the patient. The guide wire can be inserted with a reamer.

A step 208 can include drilling a bore for the post screw 102, 112. In certain embodiments, the step 208 can include drilling a first hole that is a 5 mm hole that is drilled with a depth equal to a length of the post screw 102, 112, and a second hole, 7 mm hole drilled 4 mm to 6 mm deep, can be drilled over the first hole to form the bore. A step 210 can include inserting the post screw 102, 112 into the prepared bore. The bone can be formed with a bone tool such that the bone is flush with the post screw 102, 112.

The method 200 can include a step of 212 of inserting the guide 108, 118. In particular, the attachment assembly 126, 134 of the guide 108, 118 can be placed on the star head of the post screw 102, 112. A step 214 of the method 200 can include installing the cross screw 106, 116. In particular, the cross screw 106,116 can be aligned with the guide hole 132 of the guide 108, 118 and drilled into the post screw 102, 112. The cross screw 106, 116 can then be removed, and a step 212 can include placing the cap 110, 120 onto the star head of the post screw 102, 112.

The steps of the method 200 can be repeated to install each of the metatarsal components and the toe components. After the components are joined, the implant may be tested to ensure that the toe has a smooth range of motion. When the procedure is complete, the incision is closed, and the foot is be bandaged. In some embodiments, a weight bearing or other postoperative shoe and/or brace may be used as the toe heals.

As stated above, in some embodiments, the metatarsophalangeal arthroplasty prothesis system 100 minimizes bone loss, prevents regression, is removable and/or replaceable, and does not affect the ability to perform a later fusion of the MTP joint, if needed.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods may be made within the scope of the present technology, with substantially similar results.

What is claimed is:

1. A metatarsophalangeal arthroplasty prothesis system comprising:
a metatarsal post screw including a cross hole formed therethrough;
a metatarsal cross screw configured to be disposed in the cross hole of the metatarsal post screw;
a metatarsal guide configured to be removably disposed in the metatarsal post screw and configured to guide the metatarsal cross screw into the cross hole of the metatarsal cross screw;
a metatarsal cap configured to be removably disposed in the metatarsal post screw;
a toe post screw including a cross hole formed therethrough;
a toe cross screw configured to be disposed in the cross hole of the toe post screw;
a toe guide configured to be removably disposed in the toe post screw and configured to guide the toe cross screw into the cross hole of the toe post screw; and
a toe cap configured to be removably disposed in the toe post screw.

2. The metatarsophalangeal arthroplasty prothesis system of claim 1, wherein each of the metatarsal cross screw and the toe cross screw are countersunk screws.

3. The metatarsophalangeal arthroplasty prothesis system of claim 1, wherein each of the metatarsal cross hole and the toe cross hole are threaded.

4. The metatarsophalangeal arthroplasty prothesis system of claim 1, wherein each of the metatarsal post screw and the toe post screw are self-tapping screws.

5. The metatarsophalangeal arthroplasty prothesis system of claim 1, wherein each of the metatarsal post screw and the toe post screw include a star head formed therein.

6. The metatarsophalangeal arthroplasty prothesis system of claim 5, wherein each of the metatarsal cap and the toe cap are configured to be press fit into the star heads of one of the metatarsal post screw and the toe post screw.

7. The metatarsophalangeal arthroplasty prothesis system of claim 6, wherein each of the metatarsal cap and the toe cap include a post and notch assembly configured to mate with and to provide an interference fit with the star head of one of the metatarsal post screw and the toe post screw.

8. The metatarsophalangeal arthroplasty prothesis system of claim 5, wherein each of the metatarsal guide and the toe guide are configured to be press fit into the star heads of one of the metatarsal post screw and the toe post screw.

9. The metatarsophalangeal arthroplasty prothesis system of claim 8, wherein each of the metatarsal guide and the toe guide include a post and notch assembly configured to mate with and to provide an interference fit with the star head of one of the metatarsal post screw and the toe post screw.

10. The metatarsophalangeal arthroplasty prothesis system of claim 1, wherein each of the metatarsal guide and the toe guide are substantially L-shaped including a first arm and a second arm, the first arm disposed at a 90-degree angle relative to the second arm.

11. The metatarsophalangeal arthroplasty prothesis system of claim 10, wherein the first arm of the metatarsal guide is substantially perpendicular to the metatarsal post screw and the second arm of the metatarsal guide is substantially parallel to the metatarsal post screw where the metatarsal guide is disposed on the metatarsal post screw.

12. The metatarsophalangeal arthroplasty prothesis system of claim 11, wherein the first arm of the toe guide is substantially perpendicular to the toe post screw and the second arm of the toe guide is substantially parallel to the toe post screw where the toe guide is disposed on the toe post screw.

13. The metatarsophalangeal arthroplasty prothesis system of claim 1, wherein the metatarsal guide includes a guide hole, the guide hole configured to be aligned with one of the metatarsal cross hole where the metatarsal guide is disposed on the metatarsal post screw.

14. The metatarsophalangeal arthroplasty prothesis system of claim 13, wherein the toe guide includes a guide hole, the guide hole configured to be aligned with the toe cross hole where the toe guide is disposed on the toe post screw.

15. The metatarsophalangeal arthroplasty prothesis system of claim 13, wherein the guide hole of the metatarsal guide and the guide hole of the toe guide are threaded.

16. The metatarsophalangeal arthroplasty prothesis system of claim 1, wherein the metatarsal cap has a smooth convex face.

17. The metatarsophalangeal arthroplasty prothesis system of claim 16, wherein the toe cap has a concave face configured to receive the convex face of the metatarsal cap.

18. The metatarsophalangeal arthroplasty prothesis system of claim 1, wherein each of the metatarsal post screw and the toe post screw are manufactured from titanium.

19. A metatarsophalangeal arthroplasty prothesis system comprising:
a metatarsal post screw including a cross hole formed therethrough;
a metatarsal cross screw configured to be disposed in the cross hole of the metatarsal post screw;
a metatarsal guide configured to be removably disposed in the metatarsal post screw and configured to guide the metatarsal cross screw into the cross hole of the metatarsal cross screw; and
a metatarsal cap configured to be removably disposed in the metatarsal post screw.

20. A method of installing a metatarsophalangeal arthroplasty prothesis system, comprising:
providing the first metatarsophalangeal arthroplasty prothesis system including:
a metatarsal post screw including a cross hole formed therethrough;
a metatarsal cross screw configured to be disposed in the cross hole of the metatarsal post screw;
a metatarsal guide configured to be removably disposed in the metatarsal post screw and configured to guide the metatarsal cross screw into the cross hole of the metatarsal cross screw;
a metatarsal cap configured to be removably disposed in the metatarsal post screw;
a toe post screw including a cross hole formed therethrough;
a toe cross screw configured to be disposed in the cross hole of the toe post screw;
a toe guide configured to be removably disposed in the toe post screw and configured to guide the toe cross screw into the cross hole of the toe post screw; and
a toe cap configured to be removably disposed in the toe post screw;
accessing and disarticulating the metatarsophalangeal joint
inserting a guide wire into the center and along a length of one of the metatarsal bone or the corresponding proximal phalanx;
drilling a bore for one of the metatarsal post screw and the toe post screw;
inserting the metatarsal post screw and the toe post screw into the prepared bore;
inserting one of the metatarsal guide and the toe guide;
installing the cross screw
placing one of the metatarsal cap and the toe cap.
